SEQUENCE LISTING (7 sequences)

SEQ ID NO: 1 - DNA, 1959 bp, synthetic construct
  source 1..1959: mol_type = other DNA
atggactggacctggattctctttctggtggcagccgcaactcgggtacatagcgcagaaaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaaggataagaagtttaatgggacaggcccttgccctagcgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacacctgtgcagatcaactgcacccggcccaacaacaacaccgtgaagtccattagaattggtccgggacaggcattttactacttcggagatataataggcgatatcagaatggcgcactgtcaggtgagcaaggccacctggaacgagaccctgggcaaggtggtcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgactga

SEQ ID NO: 2 - protein, 652 aa, synthetic construct
MDWTWILFLVAAATRVHSAENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPNLLSNMRGELKQCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYFGDIIGDIRMAHCQVSKATWNETLGKVVKQLRKHFGNNTIIRFAQSSGGDLEVTTHSFNCGGEFFYCNTSGLFNSTWISNTSVQGSNSTGSNDSIVLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 3 - DNA, 1905 bp, synthetic construct
  source 1..1905: mol_type = other DNA
gcagaaaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaaggataagaagtttaatgggacaggcccttgccctagcgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacacctgtgcagatcaactgcacccggcccaacaacaacaccgtgaagtccattagaattggtccgggacaggcattttactacttcggagatataataggcgatatcagaatggcgcactgtcaggtgagcaaggccacctggaacgagaccctgggcaaggtggtcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgactga

SEQ ID NO: 4 - protein, 634 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPNLLSNMRGELKQCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYFGDIIGDIRMAHCQVSKATWNETLGKVVKQLRKHFGNNTIIRFAQSSGGDLEVTTHSFNCGGEFFYCNTSGLFNSTWISNTSVQGSNSTGSNDSIVLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 5 - RNA, 2031 nt, synthetic construct
  source 1..2031: mol_type = other RNA
atggactggacatggattctgttcctggtcgctgccgctacaagagtgcattccgacgccatgaagaggggactttgctgtgttcttctgctgtgtggcgccgtgtttgttagccccgctggggccggatccaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaaggataagaagtttaatgggacaggcccttgccctagcgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacacctgtgcagatcaactgcacccggcccaacaacaacaccgtgaagtccattagaattggtccgggacaggcattttactacttcggagatataataggcgatatcagaatggcgcactgtcaggtgagcaaggccacctggaacgagaccctgggcaaggtggtcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgactga

SEQ ID NO: 6 - DNA, 1980 bp, synthetic construct
  source 1..1980: mol_type = other DNA
atggacgccatgaagaggggactttgctgtgttcttctgctgtgtggcgccgtgtttgttagccccgctggggccggatccaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacaccgatatcatttccctgtgggaccagagtctgaaaccatgtgtcaaacttactcctctgtgcgtgactctccagtgtacaaacgtgactaacaacattaccgacgacatgcggggcgagctcaagaattgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaaggataagaagtttaatgggacaggcccttgccctagcgtttcaacggtccaatgtacccacgggatcaagcccgtagtgtctacacagctcctgctgaacggcagcctggccgaagaggaggtcatgattaggagcgagaacataactaacaacgctaaaaacattctcgtccagtttaatacacctgtgcagatcaactgcacccggcccaacaacaacacccgcaagtccattagaattggtccgggacaggcattttacgctacaggagatataataggcgatatcagacaggcgcactgtaacgtgagcaaggccacctggaacgagaccctgggcaaggtggtcaaacagttgcgcaagcactttgggaacaacaccattattcggtttgccaactcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcaccttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtcttcctcggtttcctgggtgctgccggcagtacaatgggcgccgccagcatgacgctcacagtgcaggcccggaatcttcttagcggaattgtgcaacaacaaagcaatctgttgagagccccggaagctcagcaacatctgttgaagctgacagtgtggggcatcaagcagctgcaagctcgggttctggctgttgagagatacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgactga

SEQ ID NO: 7 - protein, 659 aa, synthetic construct
MDAMKRGLCCVLLLCGAVFVSPAGAGSNLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

(19) United States
(12) Patent Application Publication
(10) Pub. No.: US 2026/0207733 A1
(43) Pub. Date: Jul. 23, 2026

(54) HIV IMMUNOGENS AND METHODS OF USING SAME

(71) Applicant: The Wistar Institute of Anatomy and Biology, Philadelphia, PA (US)

(72) Inventor: Amelia Escolano, Philadelphia, PA (US)

(21) Appl. No.: 19/138,828

(22) PCT Filed: Dec. 15, 2023
(86) PCT No.: PCT/US2023/084297

§ 371 Date: Jun. 13, 2025

Related U.S. Application Data
(60) Provisional application No. 63/387,599, filed on Dec. 15, 2022.

(51) Int. Cl. A61K 39/21; A61K 39/00; A61P 37/04; C07K 14/005

(52) U.S. Cl. CPC A61K 39/21; A61P 37/04; C07K 14/005; A61K 2039/5258; A61K 2039/53; A61K 2039/575

(57) ABSTRACT

Disclosed are HIV immunogens as well as nucleic acids encoding these immunogens and methods of use of the immunogens for generating an immune response in a subject. In some embodiments, immunogens can be used for treating or preventing a human immunodeficiency type 1 (HIV-1) infection in a subject.

### CROSS REFERENCE TO RELATED APPLICATIONS

This application claims priority to U.S. Provisional Application No. 63/387,599, filed Dec. 15, 2022, which is hereby incorporated by reference herein in its entirety.

### STATEMENT REGARDING FEDERALLY SPONSORED RESEARCH OR DEVELOPMENT

This invention was made with government support under AI175470, awarded by the National Institutes of Health. The government has certain rights in the invention.

### FIELD OF THE INVENTION

This disclosure relates to immunogenic polypeptides, and specifically to mutations in immunogenic polypeptides capable of stimulating a greater immune response to human immunodeficiency virus (HIV) than native envelope glycoprotein (Env).

### BACKGROUND OF THE INVENTION

A central goal of HIV-1 vaccine development is the elicitation of broadly neutralizing antibodies (bNAbs) to the envelope glycoprotein (Env). Initial attempts at generating neutralizing antibodies by vaccination with recombinant HIV gpl20 protein analogous to some highly effective vaccines have thus far proved unsuccessful in generating protective immunity. For example, in 2009, a large multicenter, double-blind, placebo-controlled clinical study revealed that priming immunization with ALVAC-HIV (a canary pox vector vaccine expressing HIV Env genes) followed by AIDSVAX B/E booster immunizations (recombinant HIV Env gpl20 vaccine) can reduce the risk of HIV infection amongst heterosexuals by 31 percent. However, the vaccine only induced short-term protection, and there has been speculation that short-lived antibody responses to HIV Env protein might neutralize or block HIV mucosal transmission. An effective HIV-1 vaccine will likely need to induce neutralizing antibodies (NAbs) that block HIV-1 entry into human cells. To be effective, vaccine induced antibodies will have to be active against most circulating strains of HIV-1. Thus, there is a need for immunogens that can be used to elicit an immune response to pathogens, such as HIV.

### SUMMARY OF THE INVENTION

In one embodiment, the invention comprises a variant HIV Env protein or polypeptide, wherein the sequence comprises a variant which disrupts at least one N-glycosylation site. In one embodiment, the variant comprises a disruption of the N-glycosylation site at position 332 as compared to the native HIV Env sequence.

In one embodiment, the polypeptide has an amino acid sequence that is at least 75% identical to SEQ ID NO: 2 or SEQ ID NO: 4. In one embodiment, the polypeptide has an amino acid sequence that is at least 97% identical to SEQ ID NO: 2 or SEQ ID NO: 4. In one embodiment, the polypeptide comprises at least 75% of the full length of SEQ ID NO: 2 or SEQ ID NO: 4.

In one embodiment, the polypeptide binds to an antibody with an affinity having a K_{D} of about 50 μM or less.

In one embodiment, the polypeptide elicits autologous neutralizing antibodies against HIV-1 in a mammal when administered to the mammal.

In one embodiment, the invention comprises a nucleic acid molecule encoding a variant HIV Env, wherein the variant HIV Env sequence comprises a variant which disrupts at least one N-glycosylation site. In one embodiment, the variant comprises a disruption of the N-glycosylation site at amino acid residue 332 as compared to the native HIV Env sequence.

In one embodiment, the nucleic acid molecule is selected from the group consisting of DNA and RNA. In one embodiment, the nucleic acid molecule has a sequence that is at least 75% identical to SEQ ID NO: 1, SEQ ID NO: 3 or SEQ ID NO:5. In one embodiment, the nucleic acid molecule has a sequence that is at least 98% identical to a sequence selected from the group consisting of SEQ ID NO: 1, SEQ ID NO: 3 and SEQ ID NO:5. In one embodiment, the nucleic acid molecule comprises at least 75% of the full length of SEQ ID NO: 1, SEQ ID NO: 3 or SEQ ID NO:5.

In one embodiment, the invention comprises a vector comprising a nucleic acid molecule encoding a variant HIV Env protein, wherein the sequence comprises a variant which disrupts at least one N-glycosylation site. In one embodiment, the variant comprises a disruption of the N-glycosylation site at amino acid 332 as compared to the native HIV Env sequence. In one embodiment, the vector is an expression vector.

In one embodiment, the invention comprises a host cell comprising a nucleic acid molecule encoding a variant HIV Env protein, wherein the sequence comprises a variant which disrupts at least one N-glycosylation site. In one embodiment, the variant comprises a disruption of the N-glycosylation site at amino acid 332 as compared to the native HIV Env sequence.

In one embodiment, the invention comprises a composition comprising a variant HIV Env polypeptide, wherein the variant HIV Env polypeptide comprises a modification which disrupts at least one N-glycosylation site. In one embodiment, the variant HIV Env polypeptide comprises a disruption of the N-glycosylation site at amino acid 332 as compared to the native HIV Env sequence. In one embodiment, the composition comprises a protein complex or fusion protein comprising the variant HIV Env polypeptide.

In one embodiment, the invention comprises a composition comprising a nucleic acid molecule comprising a sequence encoding a variant HIV Env, wherein the variant HIV Env comprises a variant which disrupts at least one N-glycosylation site. In one embodiment, the variant comprises a disruption of the N-glycosylation site at amino acid 332 as compared to the native HIV Env sequence. In one embodiment, the composition is selected from the group consisting of a virus-like particle, a nanoparticle, and a multimer.

In one embodiment, the invention comprises an immunogenic composition for stimulating an immune response in a subject in need thereof, comprising administrating to the subject an effective amount of a variant HIV Env polypeptide or a nucleic acid molecule comprising a sequence encoding a variant HIV Env.

In one embodiment, the invention comprises a method of stimulating an immune response in a subject in need thereof, comprising administrating to the subject an effective amount of a variant HIV Env polypeptide or a nucleic acid molecule comprising a sequence encoding a variant HIV Env.

In one embodiment, the invention comprises a method of eliciting autologous neutralizing antibodies against HIV-1 in a subject in need thereof, comprising administrating the subject an effective amount of a variant HIV Env polypeptide or a nucleic acid molecule comprising a sequence encoding a variant HIV Env.

In one embodiment, the invention comprises a method of treating or preventing HIV infection in a subject in need thereof, comprising administering to the subject a therapeutically effective amount of a variant HIV Env polypeptide or a nucleic acid molecule comprising a sequence encoding a variant HIV Env.

### BRIEF DESCRIPTION OF THE DRAWINGS

FIG. 1A and FIG. 1B depict experimental results demonstrating that WIN332Q prime elicits anti-HIV antibody responses in wild type (WT) and hD3-3/JH6 mice. WIN332Q immunization elicits detectable antibody responses in wild type mice (FIG. 1A) and in transgenic mice engineered to express antibodies with long CDRH3s (FIG. 1B) (hD3-3/JH6 mice). Graphs show binding to WIN332Q in ELISAs using the serum collected from naïve and WIN332Q immunized mice at day 14.

FIG. 2 depicts experimental results demonstrating that WIN332Q prime elicits anti-HIV antibody responses that cross-react to native-like Env proteins in Rhesus Macaques. Prime immunization with WIN332Q in rhesus macaques elicits antibody responses that cross react to serially more native looking Env proteins. Graph shows ELISA binding of the serum from four macaques immunized with WIN332Q to WIN332Q and the gradually less engineered Env proteins 11MUTB, 10MUT, 7MUT, 5MUT and BG505.

FIG. 3 depicts experimental results demonstrating WIN332Q prime elicits partially specific antibody responses to the V3-glycan epitope of HIV-1 Env in Rhesus macaques. WIN332Q elicits antibody responses that target the conserved V3-glycan epitope of Env in rhesus macaques. Graph shows the level of V3-glycan specificity of the serologic antibodies elicited by WIN332Q. Values represent the ratio between the ELISA binding of the serum in the presence of an antibody against the CD4 binding site (3BNC117) and the binding of the serum in the presence of a V3-glycan antibody (PGT121).

FIG. 4 depicts experimental results demonstrating that WIN332Q prime elicits autologous neutralizing antibodies against HIV-1 in Rhesus macaques. WIN332Q immunization elicits antibodies that neutralize the autologous HIV-1 pseudovirus, BG505. Graph shows the percentage of neutralization of a BG505 T332N pseudovirus by the serum of four naïve and four WIN332Q immunized macaques in a TZM-bl assay.

### DETAILED DESCRIPTION

The invention is based on the development of a variant of the HIV envelope protein which functions as an immunogen for vaccination strategies to stimulate an immune response (e.g., HIV immune response) in a subject. In one embodiment, the immunogen of the invention is able to elicit detectable antibody responses and autologous neutralizing antibodies against HIV-1.

In one embodiment, the composition comprises a peptide comprising an amino acid sequence capable of expressing one or more HIV antigens in the subject and a pharmaceutically acceptable excipient.

In one embodiment, the composition comprises nucleotide sequences capable of expressing an HIV antigen in the subject and a pharmaceutically acceptable excipient. In one embodiment, the nucleic acid molecule comprises a promoter operably linked to a coding sequence that encodes an HIV antigen.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Adjuvant” as used herein means any molecule added to the vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. “Complement” or “complementary” as used herein means Watson-Crick (e.g., A-T/U and C-G) or Hoogsteen base pairing between nucleotides or nucleotide analogs of nucleic acid molecules. “Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Endogenous antibody” as used herein may refer to an antibody that is generated in a subject that is administered an effective dose of an antigen for induction of a humoral immune response.

“Fragment” as used herein with respect to a nucleic acid molecule refers to a portion thereof comprising consecutive nucleotide residues. Fragments of a nucleic acid can comprise at least 10%, at least 20%, at least 30%, at least 40%, at least 50%, at least 60%, at least 70%, at least 80%, at least 90% or at least 95% of the full length of the nucleic acid sequence. The fragments can be fragments of the various nucleotide sequences that encode protein fragments as set forth below.

“Fragment” with respect to polypeptide sequences refers to a portion thereof comprising consecutive amino acid residues. Fragments of a polypeptide or protein can comprise at least 10%, at least 20%, at least 30%, at least 40%, at least 50%, at least 60%, at least 70%, at least 80%, at least 90% or at least 95% of the full length of the polypeptide or protein sequence. Fragments of an polypeptide or protein can comprise at least 20 consecutive amino acids or more, at least 30 consecutive amino acids or more, at least 40 consecutive amino acids or more, at least 50 consecutive amino acids or more, at least 60 consecutive amino acids or more, at least 70 consecutive amino acids or more, at least 80 consecutive amino acids or more, at least 90 consecutive amino acids or more, at least 100 consecutive amino acids or more, at least 110 consecutive amino acids or more, at least 120 consecutive amino acids or more, at least 130 consecutive amino acids or more, at least 140 consecutive amino acids or more, at least 150 consecutive amino acids or more, at least 160 consecutive amino acids or more, at least 170 consecutive amino acids or more, at least 180 consecutive amino acids or more, at least 190 consecutive amino acids or more, at least 200 consecutive amino acids or more, at least 210 consecutive amino acids or more, at least 220 consecutive amino acids or more, at least 230 consecutive amino acids or more, or at least 240 consecutive amino acids or more of the polypeptide or protein sequence.

“Immunogenic fragment,” as used herein, means a polypeptide capable of eliciting an immune response in a mammal that cross reacts with a full-length wild type strain HIV antigen.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered.

As used herein, the term “expressible form” refers to gene constructs that contain the necessary regulatory elements operable linked to a coding sequence that encodes a protein such that when present in the cell of the individual, the coding sequence will be expressed.

gpl20 is an envelope protein from human immunodeficiency virus (HIV). The mature gpl20 wild-type polypeptides have about 500 amino acids in the primary sequence. The gpl20 is heavily N-glycosylated giving rise to an apparent molecular weight of 120 kD. The polypeptide is comprised of five conserved regions (C1-C5) and five regions of high variability (V1-V5). Exemplary sequences of wild-type gpl60 polypeptides are shown on GENBANK®, for example, Accession Nos. AAB05604 and AAD12142, which are incorporated herein by reference in their entirety as available on Jun. 29, 2010. Exemplary sequences of gpl20 polypeptides from HIV-1 DU156 are shown on GENBANK®, for example, Accession Nos. ABD83635, AA050350, and AAT91997, which are incorporated herein by reference in their entirety as available on Sep. 27, 2010. Exemplary sequences of gpl20 polypeptides from HIV-1 ZA012 are shown on GENBANK®, for example, Accession No. ACF75939, which is incorporated herein by reference in its entirety as available on Sep. 27, 2010.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immunogenic polypeptide” refers to a protein or a portion thereof that is capable of inducing an immune response in a mammal, such as a mammal infected or at risk of infection with a pathogen. Administration of an immunogenic polypeptide can lead to protective immunity against a pathogen of interest. In some examples, an immunogenic polypeptide is an antigen that is resurfaced to focus immunogenicity to a target epitope. An “immunogenic gpl20 polypeptide” is gpl20 molecule, a resurfaced gpl20 molecule, or a portion thereof capable of inducing an immune response in a mammal, such as a mammal with or without an HIV infection. Administration of an immunogenic gpl20 polypeptide that induces an immune response can lead to protective immunity against HIV.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a modified HIV envelope immunogens set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants to or is in need of being immunized with the herein described vaccine. The mammal can be a human, chimpanzee, dog, cat, horse, cow, mouse, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### DESCRIPTION

A major obstacle in the development of a HIV-1 vaccine is in inducing an immune response that overcomes the broad viral diversity of HIV-1. The trimeric HIV-1 envelope glycoprotein (ENV) remains the primary target for vaccine development as it is responsible for viral entry and is a target for broadly neutralizing antibodies (bNabs). The protein is modified by glycans that play essential roles during the viral life cycle, including protein folding, stability, infectivity, and immune invasion. Multiple immune-evasive properties hinder the ability of engineered Env proteins to induce truly useful nAb responses. These defense mechanisms include surface loops that vary in sequence in response to nAb selection pressure. These loops, and also abundant glycan structures, shield the more conserved regions of Env involved in receptor binding in regard to vaccine development, mutations in the ENV are necessary for maintaining protein stability and trimerization. Additional mutations increase accessibility to the ENV glycoprotein to broadly neutralizing antibodies (bNAbs). The current invention describes a HIV-1 envelope polypeptide that comprises mutations that elicits a polyclonal anti-HIV antibody response including antibodies that target the conserved V3-glycan epitope of Env without serial immunization.

In one embodiment, the immunogen comprises a polypeptide sequence having at least a substitution at N332 in the Env region. For example, the polypeptide may include a substitution at the position corresponding to position 332 to disrupt a glycosylation site, thereby preventing the addition of a glycan at that position. For example, this disruption of a glycosylation site can be applicable to any wild type Env, particularly HIV Env. In yet another embodiment, the disruption of a glycosylation site at position 332 in HIV Env can be applicable to all HIV strain backgrounds. As a non-limiting example, the WIN332Q immunogen was designed based on the BG505 virus (e.g., SEQ ID NO: 6 and 7). However, the invention can be applicable to in other HIV strains, clades, etc.

In one embodiment, the immunogen comprises a polypeptide having a sequence that is at least 75% identical to SEQ ID NO 2 or SEQ ID NO 4. The polypeptide may include substitutions at N332 in the Env region. For example, the polypeptide may include a substitution at the position corresponding to position 332 to remove a glycosylation site. In one example, the polypeptide includes an N332Q substitution or a conservative substitution of N332. In another example, the polypeptide, comprises the sequence set forth in SEQ ID NO: 2 or SEQ ID NO: 4.

In one embodiment, the disclosure relates to an immunogen for stimulating an immune response (e.g., HIV immune response) of a subject in need thereof. The immunogen comprises a polypeptide or nucleotide encoding the polypeptide comprising a sequence that is at least 75% identical to SEQ ID NO:2 or SEQ ID NO: 4.

In one embodiment, the polypeptide includes substitutions at positions corresponding to 332 or another conservative substitution. In one embodiment, the immunogen has the amino acid sequence of SEQ ID NO: 2 or SEQ ID NO: 4.

“Polypeptide” is used in its conventional meaning, i.e., as a sequence of amino acids. The polypeptides are not limited to a specific length of the product. Peptides, polypeptides, and proteins are included within the definition of polypeptide, and such terms can be used interchangeably herein unless specifically indicated otherwise. This term also includes post expression modifications of the polypeptide, for example, glycosylations, acetylations, phosphorylations and the like, as well as other modifications known in the art, both naturally occurring and non-naturally occurring. A polypeptide can be an entire protein or a subsequence thereof. A polypeptide “variant,” as the term is used herein, is a polypeptide that typically differs from a polypeptide specifically disclosed herein in one or more substitutions, deletions, additions and/or insertions. Such variants can be naturally occurring or can be synthetically generated, for example, by modifying one or more of the above polypeptide sequences of the disclosure and evaluating one or more biological activities of the polypeptide as described herein and/or using any of some techniques well known in the art.

For example, certain amino acids can be substituted for other amino acids in a protein structure without appreciable loss of its ability to bind other polypeptides (for example, antigens) or cells. Since it is the binding capacity and nature of a protein that defines that protein's biological functional activity, certain amino acid sequence substitutions can be made in a protein sequence, and, accordingly, its underlying DNA coding sequence, whereby a protein with like properties is obtained. It is thus contemplated that various changes can be made in the peptide sequences of the disclosed compositions, or corresponding DNA sequences that encode said peptides without appreciable loss of their biological utility or activity.

Variant sequences include those wherein conservative substitutions have been introduced by modification of polynucleotides encoding polypeptides of this disclosure. Amino acids can be classified according to physical properties and contribution to secondary and tertiary protein structure. Such conservative modifications include amino acid substitutions, additions, and deletions. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, pro line, phenylalanine, methionine), beta-branched side chains (e.g., threonine, valine, isoleucine) and aromatic side chains (e.g., tyrosine, phenylalanine, tryptophan, histidine).

“Sequence identity” or “homology” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least about 70%, at least about 75%, at least about 80%, at least about 90%, at least about 95%, at least about 98%, or at least about 99% polynucleotide or polypeptide homology with a polynucleotide or polypeptide described herein.

Polypeptide variant sequences may share 70% or more (i.e., 80%, 85%, 90%, 95%, 97%, 98%, 99% or more) sequence identity with the sequences recited in this disclosure. Polypeptide variants may also include polypeptide fragments comprising various lengths of contiguous stretches of amino acid sequences disclosed herein. Polypeptide variant sequences include at least about 5, 10, 15, 20, 30, 40, 50, 75, 100, 150, or more contiguous peptides of one or more of the sequences disclosed herein as well as all intermediate lengths therebetween.

The above-described immunogens may bind specifically to bNAbs. bNAbs are neutralizing antibodies that neutralize multiple HIV-1 viral strains. bNAbs are unique in that they target conserved epitopes of the virus. Examples of broadly neutralizing antibodies may include, without limitation, VRC26.25, PCT64-24E, VRC38.01, PG9, PGDM1400, CHOI, BG18, DH270.1, DH270.6, PGDM12, VRC41.01, PGDM21, PCDN-33A, BF520.1, VRC29.03, PGT121, 10-1074, N49-P7, N6, NC-Cowl, IOMA, CH235, CH235.12, b12, VRC01, 3BNC117, CH103, VRC-PG05, VRC34.01, ACS202, PGT151, 35022, 8ANC195, DH511.11P. Among these bNAbs, BG18, DH270.1, DH270.6, PGDM12, VRC41.01, PGDM21, PCDN-33A, BF520.1, VRC29.03, PGT121, 10-1074 broadly neutralizing antibodies bind specifically to V3 glycans.

The terms “specific binding,” “selective binding,” “selectively binds,” and “specifically binds,” refer to antibody binding to an epitope on a predetermined antigen but not to other antigens. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally.

In another aspect, this disclosure provides immunogen polypeptides that are multimerized. However, the invention is not limited to any means of multimerizing. Rather, the invention includes any means of multimerizing including but is not limited to multimerizing on a nanoparticle, on a virus-like particle (VLP) (e.g., retrovirus-like particle, HIV-like particle), and the likes. Virus-like particles, or retrovirus-like particles, in the context of the present disclosure, are membrane-surrounded structures comprising viral envelope proteins embedded within the membrane of the host cell in which they are produced, and preferably, additional viral core proteins in the VLPs. These VLPs do not contain intact viral nucleic acid, and they are non-infectious. Desirably, there is sufficient envelope protein on the surface of the VLP so that when a VLP preparation is formulated into an immunogenic composition and administered to an animal or human, an immune response (cell-mediated or humoral) is raised. Desirably, the Env protein is truncated from the carboxy terminus as compared with the naturally occurring virus envelope protein. In the context of the present invention, a “truncated” envelope protein is one which contains less than a full-length cytoplasmic domain, but retains surface antigenic determinants against which an immune response is generated, preferably a protective immune response, and it retains sufficient envelope sequence for proper precursor processing and membrane insertion. The skilled artisan can produce truncated virus envelope proteins using recombinant DNA technology and virus coding sequences, which are readily available to the public. For example, the coding sequence of a virus envelope protein can be engineered for expression in a baculovirus expression vector, for example, using a commercially available baculovirus vector, under the regulatory control of a virus promoter, with appropriate modifications of the sequence to allow functional linkage of the coding sequence to the regulatory sequence, and truncation (deletion) of the portion of the coding sequence which encodes the cytoplasmic domain of the envelope protein, again with appropriate translation stop signals and sequences which allow operable splicing of the truncated envelope and associated sequences into the vector. A specifically exemplified truncated SIV envelope protein lacks the 89 amino acids at the carboxy terminus of the naturally occurring SIV envelope protein.

In another aspect, this disclosure provides a protein complex comprising at least one above-described immunogen polypeptide multimerized via covalent or non-covalent bonding/interaction (e.g., van der Waals interactions). For example, two or more immunogen polypeptides may be cross-linked by one or more cross-linkers. Crosslinkers are reagents having reactive ends to specific functional groups (e.g., primary amines or sulfhydryls) on proteins or other molecules. Crosslinkers are capable of joining two or more molecules by a covalent bond. Crosslinkers include but are not limited to amine-to-amine crosslinkers (e.g., disuccinimidyl suberate (DSS)), amine-to-sulfhydryl crosslinkers (e.g., N-g-maleimidobutyryl-oxysuccinimide ester (GMBS)), carboxyl-to-amine crosslinkers (e.g., dicyclohexylcarbodiimide (DCC)), sulfhydryl-to-carbohydrate crosslinkers (e.g., N-b-maleimidopropionic acid hydrazide (BMPH)), sulfhydryl-to-sulfhydryl crosslinkers (e.g., 1,4-bismaieimidobutane (BMB)), photoreactive crosslinkers (e.g., N-5-azido-2-nitrobenzoyloxysuccinimide (ANB-NOS)), chemo selective ligation crosslinkers (e.g., NHS-PEG4-Azide).

### Nucleic Acids

In one embodiment, the present invention provides an immunogenic composition comprising one or more nucleic acid molecules that are capable of generating in a mammal an immune response against an HIV antigen. The present invention also provides isolated nucleic acid molecules that are capable of generating in a mammal an immune response against an HIV antigen. In one embodiment, the immunogenic composition comprises amino acid sequences for an HIV antigen having the amino acid sequence set forth in SEQ ID NO:2 or SEQ ID NO:4 or variants thereof, or fragments of SEQ ID NO:2 or SEQ ID NO:4 or variants thereof.

In one embodiment, the invention provides compositions comprising a nucleic acid molecule comprising a nucleotide sequence that encodes an HIV antigen. In one embodiment, a nucleotide sequence which encodes an HIV antigen is provided as SEQ ID NO:1, which encodes SEQ ID NO:2. In one embodiment, a nucleotide sequence which encodes an HIV antigen is provided as SEQ ID NO:3, which encodes SEQ ID NO:4.

Compositions that comprise one or more nucleotide sequence that encode an HIV antigen may be on a single plasmid. In one embodiment, a composition comprises a single plasmid that encodes an HIV antigen under a single promoter.

In one embodiment, an HIV antigen is operably linked to one or more regulatory elements. In one embodiment, a regulatory element is a leader sequence. In one embodiment, a regulatory element is a start codon. Therefore, in one embodiment, the invention relates to a nucleotide sequence as set forth in SEQ ID NO:1 or SEQ ID NO:3, or a fragment or variant thereof operably linked to a nucleotide sequence comprising a start codon at the 5′ terminus. In one embodiment, the invention relates to an amino acid sequence as set forth in SEQ ID NO:2 or SEQ ID NO:4, or a fragment or variant thereof operably linked to an amino acid encoded by a start codon (e.g., a Methionine) at the N-terminus.

In one embodiment, a regulatory element is at least one stop codon. Therefore, in one embodiment, the invention relates to a nucleotide sequence as set forth in SEQ ID NO: 1 or SEQ ID NO:3, or a fragment or variant thereof, operably linked to a nucleotide sequence comprising at least one stop codon at the 3′ terminus. In one embodiment, the nucleotide sequence is operably linked to two stop codons to increase the efficiency of translational termination.

In one embodiment, the nucleic acid molecule encodes a peptide having the amino acid sequence set forth in SEQ ID NO:2 or SEQ ID NO:4. In one embodiment, the nucleic acid molecule comprises the nucleotide sequence set forth in SEQ ID NO:1 or SEQ ID NO:3. In some embodiments, the sequence can be the nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleotide sequence set forth in SEQ ID NO:1 or SEQ ID NO:3. In other embodiments, sequence can be the nucleotide sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2 or SEQ ID NO: 4.

In some embodiments, the nucleic acid molecule comprises an RNA sequence that is a transcript from a DNA sequence having at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleotide sequence set forth in SEQ ID NO:1 or SEQ ID NO:3. In some embodiments, the nucleic acid molecule comprises an RNA sequence that encodes an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the amino acid sequence set forth in SEQ ID NO:2 or SEQ ID NO: 4.

In some embodiments, the RNA sequence can be the nucleotide sequence having at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleotide sequence set forth in SEQ ID NO:5.

In one embodiment, the HIV antigen can be a peptide having the amino acid sequence set forth in SEQ ID NO:2 or SEQ ID NO:4. In some embodiments, the antigen can have an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2 or SEQ ID NO:4.

Immunogenic fragments of SEQ ID NO:2 or SEQ ID NO:4 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of the full length of SEQ ID NO:2 or SEQ ID NO:4.

Immunogenic fragments of variants of SEQ ID NO:2 or SEQ ID NO:4 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:2 or SEQ ID NO:4 that are at least 95% identical to SEQ ID NO:2 or SEQ ID NO:4.

Some embodiments relate to immunogenic fragments of SEQ ID NO:1 or SEQ ID NO: 3 comprising at least 60%, at least 65%, at least 70%, at least 75%, at least 80%, at least 85%, at least 90%, at least 95%, at least 96%, at least 97%, at least 98% or at least 99% of the full length of SEQ ID NO:1 or SEQ ID NO:3. Immunogenic fragments can be at least 96%, at least 97% at least 98% or at least 99% identical to fragments of SEQ ID NO: 1 or SEQ ID NO:3.

In one embodiment, the nucleic acid molecule comprises a sequence at least 90% identical to SEQ ID NO:1 or SEQ ID NO:3.

In one embodiment, the nucleic acid molecule comprises an RNA sequence encoding an HIV antigen sequence described herein. For example, nucleic acid molecules may comprise an RNA sequence encoding one or more of SEQ ID NO:2, SEQ ID NO:4, a variant thereof, a fragment thereof or any combination thereof.

In one embodiment, the nucleic acid molecule comprises an immunogenic fragment of SEQ ID NO:5 comprising at least 50%, at least 60%, at least 65%, at least 70%, at least 75%, at least 80%, at least 85%, at least 90%, at least 95%, at least 96%, at least 97%, at least 98% or at least 99% of the full length of SEQ ID NO:5. Immunogenic fragments can be at least 96%, at least 97% at least 98% or at least 99% identical to fragments of SEQ ID NO:5.

### Nucleic Acid Constructs

When taken up by a cell, the DNA plasmids can remain in the cell as separate genetic material. Alternatively, RNA may be administered to the cell. It is also contemplated to provide a genetic construct as a linear minichromosome including a centromere, telomeres and an origin of replication. Genetic constructs include regulatory elements necessary for gene expression of a nucleic acid molecule. The elements include: a promoter, an initiation codon, a stop codon, and a polyadenylation signal. In addition, enhancers are often required for gene expression of the sequence that encodes the target protein or the immunomodulating protein. It is necessary that these elements be operable linked to the sequence that encodes the desired proteins and that the regulatory elements are operably in the individual to whom they are administered. Such genetic constructs may be therefore be recombinant nucleic acid molecules.

The recombinant nucleic acid molecule can include one or more recombinant nucleotide sequence constructs. The recombinant nucleotide sequence construct can include one or more components, which are described in more detail below.

The recombinant nucleotide sequence construct can include a heterologous nucleotide sequence that encodes a viral antigen, a fragment thereof, a variant thereof, or a combination thereof. The recombinant nucleotide sequence construct can also include a heterologous nucleotide sequence that encodes a protease or peptidase cleavage site. The recombinant nucleotide sequence construct can also include a heterologous nucleotide sequence that encodes an internal ribosome entry site (IRES). An IRES may be either a viral IRES or an eukaryotic IRES. The recombinant nucleotide sequence can include one or more leader sequences, in which each leader sequence encodes a signal peptide. The recombinant nucleotide sequence can include one or more promoters, one or more introns, one or more transcription termination regions, one or more initiation codons, one or more termination or stop codons, and/or one or more polyadenylation signals. The recombinant nucleotide sequence construct can also include one or more linker or tag sequences. The tag sequence can encode a hemagglutinin (HA) tag.

### Protease Cleavage Site

The recombinant nucleotide sequence construct can include heterologous nucleotide sequence encoding a protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a furin cleavage site.

### Linker Sequence

The recombinant nucleotide sequence construct can include one or more linker sequences. The linker sequence can spatially separate or link the one or more components described herein. In other embodiments, the linker sequence can encode an amino acid sequence that spatially separates or links two or more polypeptides.

### Promoter

The recombinant nucleotide sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleotide sequence construct as it is from the transcription start site in its natural setting. However, variation in this distance may be accommodated without loss of promoter function.

The promoter may be operably linked to the heterologous nucleotide sequence encoding one or more viral antigen. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### Transcription Termination Region

The recombinant nucleotide sequence construct can include one or more transcription termination regions. The transcription termination region can be downstream of the coding sequence to provide for efficient termination. The transcription termination region can be obtained from the same gene as the promoter described above or can be obtained from one or more different genes.

### Initiation Codon

The recombinant nucleotide sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### Termination Codon

The recombinant nucleotide sequence construct can include one or more termination or stop codons. The termination codon can be downstream of the coding sequence. The termination codon can be in frame with the coding sequence. The termination codon can be associated with one or more signals required for efficient translation termination. Initiation codons and stop codon are generally considered to be part of a nucleotide sequence that encodes the desired protein. However, it is necessary that these elements are functional in the mammals to whom the nucleic acid construct is administered. The initiation and termination codons must be in frame with the coding sequence.

### Polyadenylation Signal

The recombinant nucleotide sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA). Promoters and polyadenylation signals used must be functional within the cells of the individual.

### Leader Sequence

The recombinant nucleotide sequence construct can include one or more leader sequences. The leader sequence can encode a signal peptide. The signal peptide can be an immunoglobulin (Ig) signal peptide, for example, but not limited to, an IgG signal peptide and a IgE signal peptide.

In addition to regulatory elements required for DNA expression, as described above, other elements may also be included in the recombinant nucleic acid molecule. Such additional elements include enhancers. Exemplary enhancers include, but are not limited to: human actin, human myosin, human hemoglobin, human muscle creatine and viral enhancers such as those from CMV, RSV and EBV.

In order to maximize protein production, regulatory sequences may be selected which are well suited for gene expression in the cells the construct is administered into. Moreover, codons that encode said protein may be selected which are most efficiently transcribed in the host cell. One having ordinary skill in the art can produce DNA constructs that are functional in the cells.

In some embodiments, nucleic acid constructs may be provided in which the coding sequences for the proteins described herein are linked to IgE leader peptide, or such IgE leader is removed. In some embodiments, proteins described herein are linked to IgE signal peptide, or such IgE leader is removed.

In some embodiments for which protein is used, for example, one having ordinary skill in the art can, using well known techniques, produce and isolate proteins of the invention using well known techniques. In some embodiments for which protein is used, for example, one having ordinary skill in the art can, using well known techniques, inserts DNA molecules that encode a protein of the invention into a commercially available expression vector for use in well-known expression systems. For example, the commercially available plasmid pSE420 (Invitrogen, San Diego, Calif.) may be used for production of protein in Escherichia coli (E. coli). The commercially available plasmid pYES2 (Invitrogen, San Diego, Calif.) may, for example, be used for production in Saccharomyces cerevisiae strains of yeast. The commercially available MAXBAC™ complete baculovirus expression system (Invitrogen, San Diego, Calif.) may, for example, be used for production in insect cells. The commercially available plasmid pcDNA or pcDNA3 (Invitrogen, San Diego, Calif.) may, for example, be used for production in mammalian cells such as Chinese hamster ovary (CHO) cells. One having ordinary skill in the art can use these commercial expression vectors and systems or others to produce protein by routine techniques and readily available starting materials. (See e.g., Sambrook et al., Molecular Cloning a Laboratory Manual, Second Ed. Cold Spring Harbor Press (1989)). Thus, the desired proteins can be prepared in both prokaryotic and eukaryotic systems, resulting in a spectrum of processed forms of the protein.

### Vector

The recombinant nucleotide sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleotide sequence construct is produced by the cellular-transcription and translation machinery ribosomal complexes. The one or more vectors can express large amounts of stable messenger RNA, and therefore proteins.

### Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleotide sequence construct may be chimeric, meaning that at least one of its components is heterologous with respect to at least one of its other components.

### Plasmid

The one or more vectors can be a plasmid. The plasmid may be useful for transfecting cells with the recombinant nucleotide sequence construct. The plasmid may be useful for introducing the recombinant nucleotide sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### RNA

In one embodiment, the nucleic acid is an RNA molecule. In one embodiment, the RNA molecule is transcribed from a DNA sequence described herein. For example, in some embodiments, the RNA molecule is encoded by a DNA sequence at least 50% identical to SEQ ID NO: 1 or SEQ ID NO:3. In another embodiment, the nucleotide sequence comprises an RNA sequence transcribed by a DNA sequence encoding a polypeptide sequence of SEQ ID NO:2 or SEQ ID NO:4, or a variant thereof or a fragment thereof. In another embodiment, the nucleotide sequence comprises an RNA set forth in SEQ ID NO:5, or a variant thereof or a fragment thereof. The RNA may be plus-stranded. Accordingly, in some embodiments, the RNA molecule can be translated by cells without needing any intervening replication steps such as reverse transcription. An RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. An RNA molecule may have a 3′ poly-A tail. It may also include a poly-A polymerase recognition sequence (e.g., AAUAAA) near its 3′ end. A RNA molecule useful with the invention may be single-stranded. An RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell. In one embodiment, the RNA is a nucleoside-modified RNA. Nucleoside-modified RNA have particular advantages over non-modified RNA, including for example, increased stability, low or absent innate immunogenicity, and enhanced translation.

### 1) Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleotide sequence construct. Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleotide sequence construct. The LEC may be any linear DNA devoid of any phosphate backbone. The LEC may not contain any antibiotic resistance genes and/or a phosphate backbone. The LEC may not contain other nucleotide sequences unrelated to the desired gene expression. The LEC may be derived from any plasmid capable of being linearized. The plasmid may be capable of expressing the heavy chain polypeptide and/or light chain polypeptide encoded by the recombinant nucleotide sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleotide sequence construct.

The LEC can be perM2. The LEC can be pcrNP. pcrNP and pcrMR can be derived from pNP (Puerto Rico/34) and pM2 (New Caledonia/99), respectively.

### m) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### n) Method of Preparing the Vector

Provided herein is a method for preparing the one or more vectors in which the recombinant nucleotide sequence construct has been placed. After the final subcloning step, the vector can be used to inoculate a cell culture in a large scale fermentation tank, using known methods in the art.

### Vaccines and Immunogenic Compositions

Immunogenic compositions, such as vaccines, are provided comprising an optimized consensus sequence, an optimized consensus-encoded antigen, a fragment thereof, a variant thereof, or a combination thereof. The immunogenic composition can significantly induce an immune response of a subject administered with the immunogenic composition against the HIV antigen. The vaccine may comprise a plurality of the nucleic acid molecules, or combinations thereof. The vaccine may be provided to induce a therapeutic or prophylactic immune response.

The immunogenic composition can be a DNA vaccine, an RNA vaccine, a peptide vaccine, or a combination vaccine. The vaccine can include an optimized consensus nucleotide sequence encoding an antigen. The nucleotide sequence can be DNA, RNA, cDNA, a variant thereof, a fragment thereof, or a combination thereof. The nucleotide sequence can also include additional sequences that encode linker, leader, or tag sequences that are linked to the antigen by a peptide bond. The peptide vaccine can include an antigen, a variant thereof, a fragment thereof, or a combination thereof. The combination DNA and peptide vaccine can include the above described optimized consensus nucleotide sequence and the encoded antigen.

The vaccine can be a DNA vaccine. DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome.

The vaccine can be an RNA of the one or more HIV antigens. The RNA vaccine can be introduced into the cell.

The vaccine can use recombinant vectors to deliver antigen, subunit vaccines, and glycoprotein vaccines, for example, but not limited, the vaccines described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

The vaccine of the present invention can have features required of effective vaccines such as being safe so that the vaccine itself does not cause illness or death; being protective against illness; inducing protective T cell responses; and providing ease of administration, few side effects, biological stability, and low cost per dose.

Provided herein is an immunogenic composition capable of generating in a mammal an immune response against HIV. The immunogenic composition may comprise each plasmid as discussed above. The immunogenic composition may comprise a plurality of the plasmids, or combinations thereof. The immunogenic composition may be provided to induce a therapeutic or prophylactic immune response. In one embodiment, the composition of the invention can induce immunization after one immunization.

Immunogenic compositions may be used to deliver nucleic acid molecules that encode one or more consensus HIV antigen. Immunogenic compositions are preferably compositions comprising plasmids.

The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

Exemplary molecules that can be included in the immunogenic composition of the invention include:

| SEQ ID NO: | Type | Description |
|---|---|---|
| 1 | Nucleotide | WIN332Q (with signal sequence) |
| 2 | Amino Acid | WIN332Q (with signal sequence) |
| 3 | Nucleotide | WIN332Q (without signal sequence) |
| 4 | Amino Acid | WIN332Q (without signal sequence) |
| 5 | RNA | WIN332Q |

In one embodiment, the nucleic acid molecule comprises an optimized nucleic acid sequence. The optimized sequence can comprise a consensus sequence and/or modification(s) for improved expression. Modification can include codon optimization, RNA optimization, addition of a kozak sequence for increased translation initiation, and/or the addition of an immunoglobulin leader sequence to increase immunogenicity. The HIV antigen encoded by the optimized sequence can comprise a signal peptide such as an immunoglobulin signal peptide, for example, but not limited to, an immunoglobulin E (IgE) or immunoglobulin (IgG) signal peptide. The HIV antigen encoded by the optimized sequence can be designed to elicit stronger cellular and/or humoral immune responses than a corresponding native antigen.

The immunogenic composition can induce an immune response in the subject administered the composition. The induced immune response can be specific for at least one HIV antigen.

The immunogenic composition can induce a humoral immune response in the subject administered the immunogenic composition. The induced humoral immune response can be specific for at least one HIV antigen. The induced humoral immune response can be reactive with at least one HIV antigen related to an administered optimized encoded antigen. The humoral immune response can be induced in the subject administered the immunogenic composition by about 1.5-fold to about 16-fold, about 2-fold to about 12-fold, or about 3-fold to about 10-fold. The humoral immune response can be induced in the subject administered the immunogenic composition by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic composition of the invention.

The humoral immune response induced by the immunogenic composition can include an increased level of one or more of IgG, IgM, IgA, and the likes associated with the subject administered the immunogenic composition as compared to a subject not-administered the immunogenic composition. These IgG antibodies can be specific for at least one HIV antigen genetically related to an administered optimized encoded antigen. These IgG antibodies can be reactive with at least one HIV antigen genetically related to an administered optimized encoded antigen. The level of IgG antibody associated with the subject administered the immunogenic composition can be increased by about 1.5-fold to about 16-fold, about 2-fold to about 12-fold, or about 3-fold to about 10-fold as compared to the subject not-administered the immunogenic composition. The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic composition.

The immunogenic composition can induce a cellular immune response in the subject administered the immunogenic composition. The induced cellular immune response can be specific for at least one HIV antigen genetically related to an administered optimized encoded antigen. The induced cellular immune response can be reactive to at least one HIV antigen genetically related to an administered optimized consensus-encoded antigen. The induced cellular immune response can include eliciting a CD8+ T cell response. The elicited CD8+ T cell response can be reactive with at least one HIV antigen genetically related to an administered optimized encoded antigen. The elicited CD8+ T cell response can be polyfunctional. The induced cellular immune response can include eliciting a CD8+ T cell response, in which the CD8+ T cells produce interferon-gamma (IFN-γ), tumor necrosis factor alpha (TNF-α), interleukin-2 (IL-2), or a combination of IFN-γ and TNF-α.

The induced cellular immune response can include an increased CD8+ T cell response associated with the subject administered the immunogenic composition as compared to the subject not-administered the immunogenic composition. The CD8+ T cell response associated with the subject administered the immunogenic composition can be increased by about 2-fold to about 30-fold, about 3-fold to about 25-fold, or about 4-fold to about 20-fold as compared to the subject not-administered the immunogenic composition. The CD8+ T cell response associated with the subject administered the immunogenic composition can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to a subject not administered the immunogenic composition.

The induced cellular immune response can include an increased frequency of CD107a/IFNγ/T-bet triple-positive CD8 T cells that are reactive against the native antigen. The frequency of CD107a/IFNγ/T-bet triple-positive CD8 T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to a subject not-administered the immunogenic composition.

The induced cellular immune response can include an increased frequency of CD107a/IFNγ double-positive CD8 T cells that are reactive against the native antigen. The frequency of CD107a/IFNγ double-positive CD8 T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to a subject not-administered the immunogenic composition.

The cellular immune response induced by the immunogenic composition can include eliciting a CD4+ T cell response. The elicited CD4+ T cell response can be reactive with the native antigen genetically related to the optimized consensus antigen. The elicited CD4+ T cell response can be polyfunctional. The induced cellular immune response can include eliciting a CD4+ T cell response, in which the CD4+ T cells produce IFN-γ, TNF-α, IL-2, or a combination of IFN-γ and TNF-α.

The induced cellular immune response can include an increased frequency of CD4+ T cells that produce IFN-γ. The frequency of CD4+IFN-γ+ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to a subject not-administered the immunogenic composition.

The induced cellular immune response can include an increased frequency of CD4+ T cells that produce TNF-α. The frequency of CD4+ TNF-α+ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to a subject not-administered the immunogenic composition.

The induced cellular immune response can include an increased frequency of CD4+ T cells that produce both IFN-γ and TNF-α. The frequency of CD4+IFN-γ+ TNF-α+ associated with the subject administered the immunogenic composition can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to a subject not administered the immunogenic composition.

In one embodiment, the immunogenic composition can be a DNA vaccine, an RNA vaccine, a peptide vaccine, or a combination thereof. The immunogenic composition can include a nucleic acid molecule comprising a sequence encoding the HIV antigen in the form of a self-assembling nanoparticle. The nucleic acid sequence can be DNA, RNA, cDNA, a variant thereof, a fragment thereof, or a combination thereof. The nucleic acid sequence can also include additional sequences that encode linker, leader, or tag sequences that are linked to the HIV antigen by a peptide bond.

In one embodiment, the immunogen can be used as a priming immunogen. In another embodiment, the immunogen can be used as the second, third, fourth, fifth, sixth immunogen of a series.

### Other Components of the Composition

In some embodiments, the immunogenic composition of the invention further includes a pharmaceutically acceptable excipient. A pharmaceutically acceptable excipient can include such functional molecules as vehicles, adjuvants, carriers or diluents, which are known and readily available to the public. Preferably, the pharmaceutically acceptable excipient is an adjuvant or transfection facilitating agent. In some embodiments, the nucleic acid molecule, or DNA plasmid, is delivered to the cells in conjunction with administration of a polynucleotide function enhancer or a genetic vaccine facilitator agent (or transfection facilitating agent). Polynucleotide function enhancers are described in U.S. Pat. Nos. 5,593,972, 5,962,428 and International Application Serial Number PCT/US94/00899 filed Jan. 26, 1994, which are each incorporated herein by reference. Genetic vaccine facilitator agents are described in US. Ser. Number 021,579 filed Apr. 1, 1994, which is incorporated herein by reference. The transfection facilitating agent can be administered in conjunction with nucleic acid molecules as a mixture with the nucleic acid molecule or administered separately simultaneously, before or after administration of nucleic acid molecules. Examples of transfection facilitating agents includes surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

In some embodiments of the present invention, the immunogenic compositions can further include an adjuvant. In some embodiments, the adjuvant is selected from the group consisting of: alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-1, MIP-1-alpha, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFKB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof. In some preferred embodiments, the adjuvant is selected from IL-12, IL-15, CTACK, TECK, or MEC.

The immunogenic compositions according to the present invention are formulated according to the mode of administration to be used. In cases where DNA plasmid vaccines are injectable compositions, they are sterile, and/or pyrogen free and/or particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. In some embodiments, a stabilizing agent that allows the formulation to be stable at room or ambient temperature for extended periods of time, such as LGS or other polycations or polyanions is added to the formulation.

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Methods of Delivery of the Composition

The present invention also relates to methods of delivering the composition to the subject in need thereof. The method of delivery can include, administering the composition to the subject. The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Method of Treatment

Also provided herein is a method of treating, protecting against, and/or preventing disease in a subject in need thereof by inducing an immune response against a viral antigen in the subject. In certain embodiments, the invention provides a method of treating, protecting against, and/or preventing at least one of an HIV virus infection or an HIV associated pathology in a subject.

The method can include administering an immunogenic composition of the invention to the subject. Administration of the composition to the subject can be done using the method of delivery described above.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety). The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

### Kit

Provided herein is a kit, which can be used for treating a subject using the method of vaccination described above. In one embodiment, the kit can comprise the vaccine. In one embodiment, the kit can comprise a nucleic acid molecule encoding a modified HIV envelope immunogen of the invention.

The kit can also comprise instructions for carrying out the vaccination method described above and/or how to use the kit. Instructions included in the kit can be affixed to packaging material or can be included as a package insert. While instructions are typically written or printed materials, they are not limited to such. Any medium capable of storing instructions and communicating them to an end user is contemplated by this disclosure. Such media include, but are not limited to, links to websites, QR codes, electronic storage media (e.g., magnetic discs, tapes, cartridges), optical media (e.g., CD ROM), and the like. As used herein, the term “instructions” can include the address of an internet site which provides instructions.

### Isolating Antibodies

In one embodiment, the composition of the invention can be used as a bait or otherwise referred to as a tag in strategies to isolate desired antibodies. In one embodiment, the bait can be used to isolate anti-HIV bNABs from animals and humans. In one embodiment, the polypeptide of the invention is modified to be used as a bait. The invention should not be limited to a particular modification suitable for use as a bait. Rather, any modification can be used such as attaching the polypeptide of the invention to a tag.

In one embodiment, the polypeptide of the invention can be fused or attached to an affinity tag. The affinity tag allows for separation of the desired antibody from a mixture. In one embodiment, the affinity tag includes but is not limited to an avidin molecule or an antibody that binds to hapten and the like.

In another embodiment, the polypeptide of the invention can be fused or attached to a suitable precipitating agent. A suitable precipitating agent can be, for example, a polymer. Suitable polymers can be, for example, biopolymers, synthetic polymers, modified biopolymers or some combination thereof. Suitable biopolymers can be, for example, polysaccharides and polypeptides. In various embodiments, the precipitating agent is a biopolymer. In certain embodiments, the biopolymer is a polysaccharide. In a further embodiment, the polysaccharide is selected from the group consisting of starch, dextran, cellulose, chitin, xantum gum, glycosaminoglycan and combinations thereof. In a still further aspect, the glycosaminoglycan is selected from the group consisting of heparin, heparin sulfate, chondroitin sulfate, and combinations thereof. In other embodiments, the biopolymer is a polypeptide. In certain embodiments, the polypeptide is selected from the group consisting of albumin, collagen, fibrinogen, immunoglobulin, and combinations thereof.

In one embodiment, the polypeptide of the invention can be attached to a precipitating agent by non-covalent bonding. A suitable non-covalent method of attaching the polypeptide to the precipitating agent may use, for example, the binding of biotin and avidin. Avidin is a tetrameric protein that binds tightly to four biotin molecules. Biotin, also called vitamin B7, can be attached by biotinylation using standard chemical kits. Similarly, streptavidin and avidin can be ligated using a protein chemistry ligation kit. Streptavidin can be linked to the precipitating agent.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: WIN332Q Immunization Elicits Anti-HIV Antibody Responses

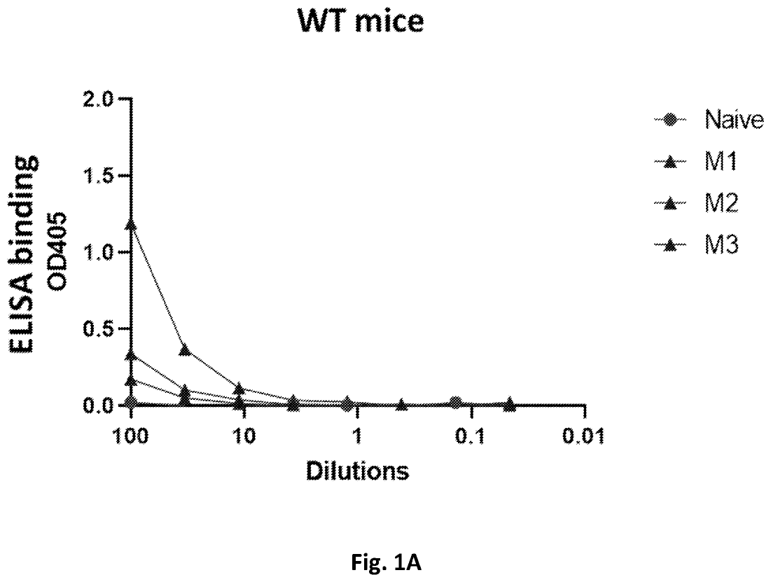

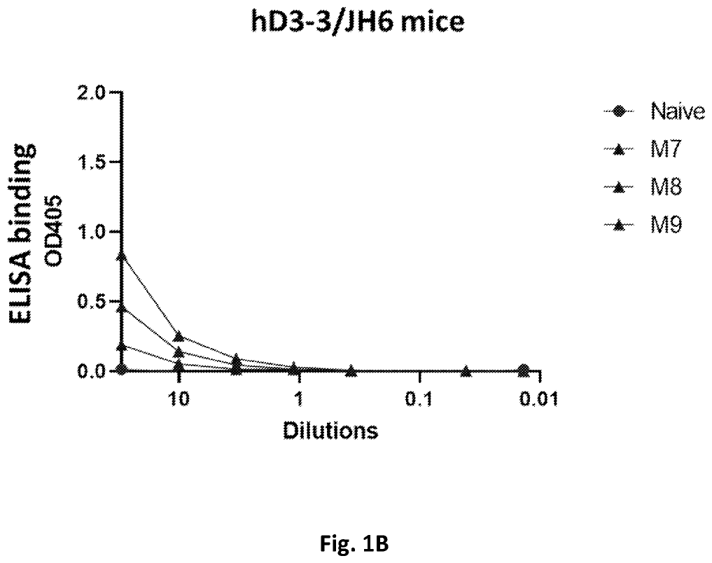

Broadly neutralizing antibodies (bNAbs) protect against HIV-1 infection, suggesting that a vaccine that elicits them would be effective. However, one of the major hurdles is that vaccination does not elicit bNAbs, in part, because B-cells expressing germline bNAb precursors do not respond to native-like HIV-1 envelope (Env) antigens. Accordingly, this disclosure provides immunogens that facilitate recognition of the V3-glycan patch on HIV-1 Env while concealing non-conserved immunodominant regions. This disclosure demonstrates that mouse, and Rhesus macaque immunizations with WIN332Q elicited serologic responses targeting the V3-glycan patch. Thus, the disclosed immunogen provides a suitable priming immunogen for sequential vaccination strategies to stimulate an immune response (e.g., HIV immune response) in a subject. WIN332Q prime elicits anti-HIV antibody responses in WT and hD3-3/JH6 mice. WIN332Q immunization elicits detectable antibody responses in both wild type mice (FIG. 1A) and in transgenic mice engineered to express antibodies with and long CDRH3s (hD3-3/JH6 mice kindly provided by Dr. Frederick Alt) (FIG. 1B). Graphs show binding to WIN332Q in ELISAs using the serum collected from naïve and WIN332Q immunized mice at day 14.

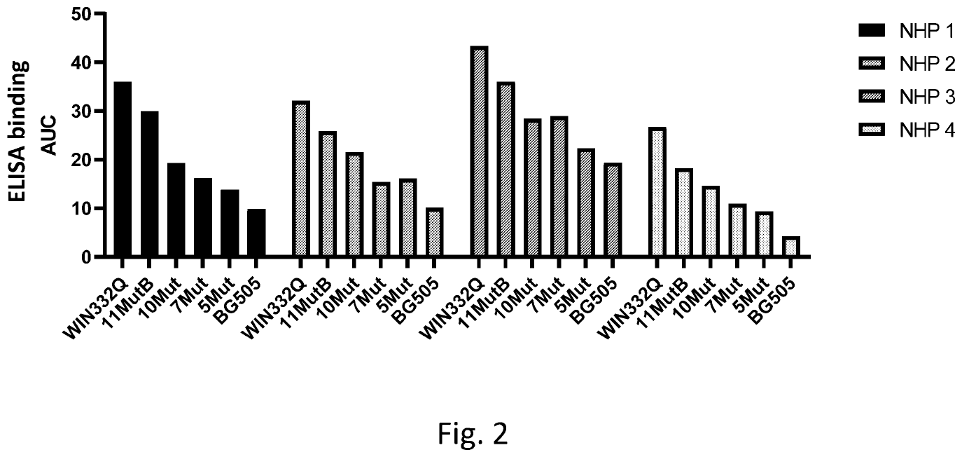

The experimental results depicted in FIG. 2 demonstrate that WIN332Q prime elicits anti-HIV antibody responses that cross-react to native-like Envs in Rhesus Macaques (FIG. 2). Prime immunization with WIN332Q in rhesus macaques elicits antibody responses that cross react to serially more native looking Envs. Graph shows ELISA binding of the serum from four macaques immunized with WIN332Q to WIN332Q and the gradually less engineered Env proteins 11MUTB, 10MUT, 7MUT, 5MUT and BG505.

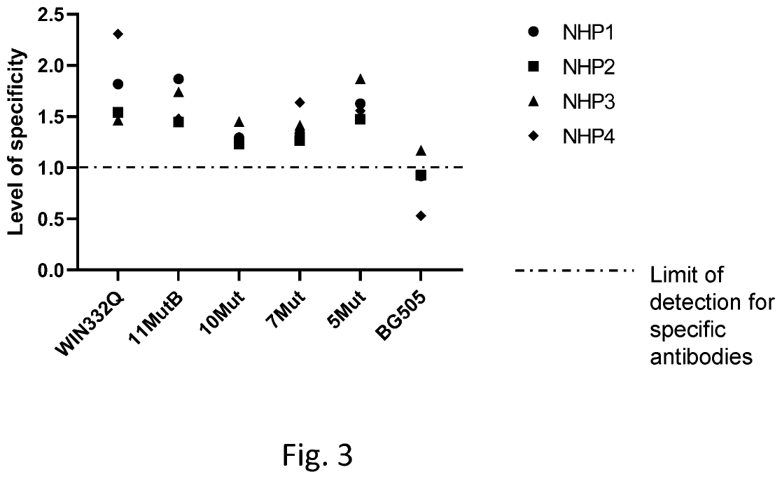

WIN332Q prime elicits partially specific antibody responses to the V3-glycan epitope in Rhesus macaques. WIN332Q elicits antibody responses that target the conserved V3-glycan epitope of Env in rhesus macaques (FIG. 3). Graph shows the level of V3-glycan specificity of the serologic antibodies elicited by WIN332Q. Values represent the ratio between the ELISA binding of the serum in the presence of an antibody against the CD4 binding site (3BNC117) and the binding of the serum in the presence of a V3-glycan antibody (PGT121).

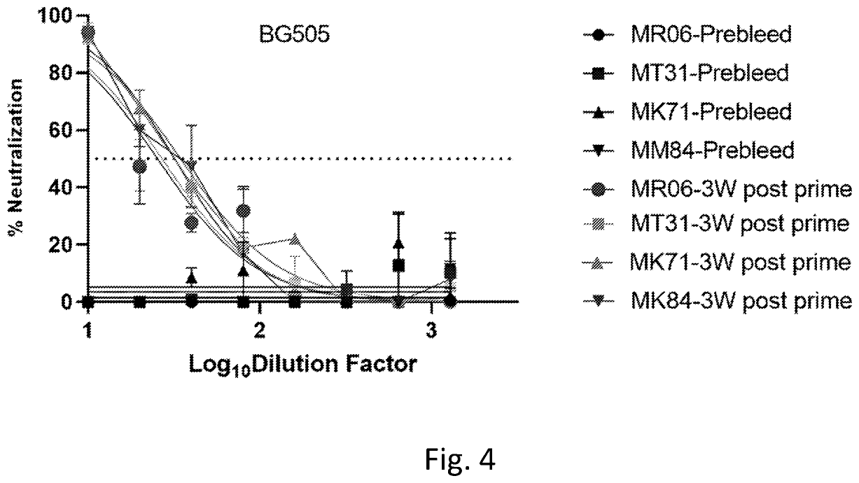

WIN332Q prime elicits autologous neutralizing antibodies against HIV-1 in Rhesus macaques. WIN332Q immunization elicits antibodies that neutralize the autologous HIV-1 pseudovirus, BG505. Data show the percentage of neutralization of a BG505 T332N pseudovirus by the serum of four naïve and WIN332Q immunized macaques in a TZM-bl assay (FIG. 4).

### Example 2: Sequences

SEQ ID: 1 (with signal peptide)
ATGGACTGGACCTGGATTCTCTTTCTGGTGGCAGCCGCAACTCGG
GTACATAGCGCAGAAAACCTGTGGGTCACTGTGTATTATGGTGTG
CCAGTGTGGAAGGATGCAGAGACAACACTCTTTTGCGCCTCCGAC
GCTAAAGCATACGAAACGGAGAAGCACAACGTGTGGGCGACCCAT
GCCTGTGTCCCTACAGACCCTAACCCTCAGGAAATTCATCTTGAA
AATGTCACAGAAGAGTTTAACATGTGGAAAAACAACATGGTGGAA
CAGATGCACGAGGATATCATTTCCCTGTGGGACCAGAGTCTGAAA
CCATGTGTCAAACTTACTCCTCTGTGCGTGACTCTCCAGTGTACA
AACTACGCACCCAACCTTTTGAGTAATATGCGGGGCGAGCTCAAG
CAGTGCAGTTTCAATATGACAACCGAATTGAGAGACAAAAAACAG
AAAGTATACTCCCTCTTCTACCGGCTGGACGTGGTGCAGATCAAT
GAGAACCAAGGAAATAGAAGCAACAACAGTAACAAGGAATACCGG
CTCATAAATTGCAATACCAGCGCTATTACGCAGGCTTGCCCTAAG
GTGAGCTTTGAGCCAATCCCGATACATTATTGTGCCCCGGCAGGC
TTCGCTATACTGAAATGCAAGGATAAGAAGTTTAATGGGACAGGC
CCTTGCCCTAGCGTTTCAACGGTCCAATGTACCCACGGGATCAAG
CCCGTAGTGTCTACACAGCTCCTGCTGAACGGCAGCCTGGCCGAA
GAGGAGGTCATAATTAGGAGCGAGAACATAACTAACAACGCTAAA
AACATTCTCGTCCAGCTCAATACACCTGTGCAGATCAACTGCACC
CGGCCCAACAACAACACCGTGAAGTCCATTAGAATTGGTCCGGGA
CAGGCATTTTACTACTTCGGAGATATAATAGGCGATATCAGAATG
GCGCACTGTCAGGTGAGCAAGGCCACCTGGAACGAGACCCTGGGC
AAGGTGGTCAAACAGTTGCGCAAGCACTTTGGGAACAACACCATT
ATTCGGTTTGCCCAGTCTTCCGGCGGCGACCTTGAAGTGACCACT
CATAGCTTCAACTGTGGAGGGGAGTTTTTCTATTGCAATACATCA
GGCCTGTTCAACTCTACATGGATCTCAAATACCAGTGTCCAGGGG
TCAAATTCCACCGGTAGCAACGACAGCATCGTCTTGCCTTGTCGA
ATCAAGCAGATCATTAATATGTGGCAGAGGATTGGTCAGGCCATG
TACGCACCTCCAATACAGGGAGTCATTCGGTGCGTCAGCAATATT
ACTGGATTGATCCTCACCAGAGATGGCGGGAGTACCAATAGCACT
ACCGAAACTTTCCGCCCAGGAGGAGGCGACATGCGGGATAATTGG
AGATCAGAGCTGTATAAGTATAAGGTGGTGAAAATTGAACCCCTG
GGAGTGGCGCCAACTAGATGTAAACGGCGAGTGGTTGGCCGGAGA
CGGCGGCGGAGAGCAGTGGGGATTGGCGCTGTCTCACTCGGTTTC
CTGGGTGCTGCCGGCAGTACAATGGGCGCCGCCAGCATGACGCTC
ACAGTGCAGGCCCGGAATCTTCTTAGCGGAATTGTGCAACAACAA
AGCAATCTGTTGAGAGCCCCGGAACCGCAGCAACATCTGTTGAAG
GACACACATTGGGGCATCAAGCAGCTGCAAGCTCGGGTTCTGGCT
GTTGAGCATTACCTGAGAGACCAACAGCTGCTGGGCATATGGGGA
TGCTCAGGAAAACTGATCTGCTGCACCAATGTCCCATGGAACAGC
TCATGGTCAAACAGGAACCTGAGCGAGATCTGGGATAACATGACC
TGGTTGCAGTGGGACAAAGAAATTAGCAATTACACACAGATCATC
TACGGCCTCCTGGAGGAAAGCCAGAATCAGCAGGAGAAAAATGAG
CAGGATCTGCTTGCCCTTGACTGA
SEQ ID: 2 (with signal peptide)
MDWTWILFLVAAATRVHSAENLWVTVYYGVPVWKDAETTLFCASD
AKAYETEKHNVWATHACVPTDPNPQEIHLENVTEEFNMWKNNMVE
QMHEDIISLWDQSLKPCVKLTPLCVTLQCTNYAPNLLSNMRGELK
QCSFNMTTELRDKKQKVYSLFYRLDVVQINENQGNRSNNSNKEYR
LINCNTSAITQACPKVSFEPIPIHYCAPAGFAILKCKDKKFNGTG
PCPSVSTVQCTHGIKPVVSTQLLLNGSLAEEEVIIRSENITNNAK
NILVQLNTPVQINCTRPNNNTVKSIRIGPGQAFYYFGDIIGDIRM
AHCQVSKATWNETLGKVVKQLRKHFGNNTIIRFAQSSGGDLEVTT
HSFNCGGEFFYCNTSGLFNSTWISNTSVQGSNSTGSNDSIVLPCR
IKQIINMWQRIGQAMYAPPIQGVIRCVSNITGLILTRDGGSTNST
TETFRPGGGDMRDNWRSELYKYKVVKIEPLGVAPTRCKRRVVGRR
RRRRAVGIGAVSLGFLGAAGSTMGAASMTLTVQARNLLSGIVQQQ
SNLLRAPEPQQHLLKDTHWGIKQLQARVLAVEHYLRDQQLLGIWG
CSGKLICCTNVPWNSSWSNRNLSEIWDNMTWLQWDKEISNYTQII
YGLLEESQNQQEKNEQDLLALD
SEQ ID NO 3 (without signal peptide)
GCAGAAAACCTGTGGGTCACTGTGTATTATGGTGTGCCAGTGTGG
AAGGATGCAGAGACAACACTCTTTTGCGCCTCCGACGCTAAAGCA
TACGAAACGGAGAAGCACAACGTGTGGGCGACCCATGCCTGTGTC
CCTACAGACCCTAACCCTCAGGAAATTCATCTTGAAAATGTCACA
GAAGAGTTTAACATGTGGAAAAACAACATGGTGGAACAGATGCAC
GAGGATATCATTTCCCTGTGGGACCAGAGTCTGAAACCATGTGTC
AAACTTACTCCTCTGTGCGTGACTCTCCAGTGTACAAACTACGCA
CCCAACCTTTTGAGTAATATGCGGGGCGAGCTCAAGCAGTGCAGT
TTCAATATGACAACCGAATTGAGAGACAAAAAACAGAAAGTATAC
TCCCTCTTCTACCGGCTGGACGTGGTGCAGATCAATGAGAACCAA
GGAAATAGAAGCAACAACAGTAACAAGGAATACCGGCTCATAAAT
TGCAATACCAGCGCTATTACGCAGGCTTGCCCTAAGGTGAGCTTT
GAGCCAATCCCGATACATTATTGTGCCCCGGCAGGCTTCGCTATA
CTGAAATGCAAGGATAAGAAGTTTAATGGGACAGGCCCTTGCCCT
AGCGTTTCAACGGTCCAATGTACCCACGGGATCAAGCCCGTAGTG
TCTACACAGCTCCTGCTGAACGGCAGCCTGGCCGAAGAGGAGGTC
ATAATTAGGAGCGAGAACATAACTAACAACGCTAAAAACATTCTC
GTCCAGCTCAATACACCTGTGCAGATCAACTGCACCCGGCCCAAC
AACAACACCGTGAAGTCCATTAGAATTGGTCCGGGACAGGCATTT
TACTACTTCGGAGATATAATAGGCGATATCAGAATGGCGCACTGT
CAGGTGAGCAAGGCCACCTGGAACGAGACCCTGGGCAAGGTGGTC
AAACAGTTGCGCAAGCACTTTGGGAACAACACCATTATTCGGTTT
GCCCAGTCTTCCGGCGGCGACCTTGAAGTGACCACTCATAGCTTC
AACTGTGGAGGGGAGTTTTTCTATTGCAATACATCAGGCCTGTTC
AACTCTACATGGATCTCAAATACCAGTGTCCAGGGGTCAAATTCC
ACCGGTAGCAACGACAGCATCGTCTTGCCTTGTCGAATCAAGCAG
ATCATTAATATGTGGCAGAGGATTGGTCAGGCCATGTACGCACCT
CCAATACAGGGAGTCATTCGGTGCGTCAGCAATATTACTGGATTG
ATCCTCACCAGAGATGGCGGGAGTACCAATAGCACTACCGAAACT
TTCCGCCCAGGAGGAGGCGACATGCGGGATAATTGGAGATCAGAG
CTGTATAAGTATAAGGTGGTGAAAATTGAACCCCTGGGAGTGGCG
CCAACTAGATGTAAACGGCGAGTGGTTGGCCGGAGACGGCGGCGG
AGAGCAGTGGGGATTGGCGCTGTCTCACTCGGTTTCCTGGGTGCT
GCCGGCAGTACAATGGGCGCCGCCAGCATGACGCTCACAGTGCAG
GCCCGGAATCTTCTTAGCGGAATTGTGCAACAACAAAGCAATCTG
TTGAGAGCCCCGGAACCGCAGCAACATCTGTTGAAGGACACACAT
TGGGGCATCAAGCAGCTGCAAGCTCGGGTTCTGGCTGTTGAGCAT
TACCTGAGAGACCAACAGCTGCTGGGCATATGGGGATGCTCAGGA
AAACTGATCTGCTGCACCAATGTCCCATGGAACAGCTCATGGTCA
AACAGGAACCTGAGCGAGATCTGGGATAACATGACCTGGTTGCAG
TGGGACAAAGAAATTAGCAATTACACACAGATCATCTACGGCCTC
CTGGAGGAAAGCCAGAATCAGCAGGAGAAAAATGAGCAGGATCTG
CTTGCCCTTGACTGA
SEQ ID NO 4: (without signal peptide)
AENLWVTVYYGVPVWKDAETTLFCASDAKAYETEKHNVWATHACV
PTDPNPQEIHLENVTEEFNMWKNNMVEQMHEDIISLWDQSLKPCV
KLTPLCVTLQCTNYAPNLLSNMRGELKQCSFNMTTELRDKKQKVY
SLFYRLDVVQINENQGNRSNNSNKEYRLINCNTSAITQACPKVSF
EPIPIHYCAPAGFAILKCKDKKFNGTGPCPSVSTVQCTHGIKPVV
STQLLLNGSLAEEEVIIRSENITNNAKNILVQLNTPVQINCTRPN
NNTVKSIRIGPGQAFYYFGDIIGDIRMAHCQVSKATWNETLGKVV
KQLRKHFGNNTIIRFAQSSGGDLEVTTHSFNCGGEFFYCNTSGLF
NSTWISNTSVQGSNSTGSNDSIVLPCRIKQIINMWQRIGQAMYAP
PIQGVIRCVSNITGLILTRDGGSTNSTTETFRPGGGDMRDNWRSE
LYKYKVVKIEPLGVAPTRCKRRVVGRRRRRRAVGIGAVSLGFLGA
AGSTMGAASMTLTVQARNLLSGIVQQQSNLLRAPEPQQHLLKDTH
WGIKQLQARVLAVEHYLRDQQLLGIWGCSGKLICCTNVPWNSSWS
NRNLSEIWDNMTWLQWDKEISNYTQIIYGLLEESQNQQEKNEQDL
LALD
SEQ ID NO 5 (mRNA sequence of WIN332Q)
AUGGACUGGACAUGGAUUCUGUUCCUGGUCGCUGCCGCUACAAGA
GUGCAUUCCGACGCCAUGAAGAGGGGACUUUGCUGUGUUCUUCUG
CUGUGUGGCGCCGUGUUUGUUAGCCCCGCUGGGGCCGGAUCCAAC
CUGUGGGUCACUGUGUAUUAUGGUGUGCCAGUGUGGAAGGAUGCA
GAGACAACACUCUUUUGCGCCUCCGACGCUAAAGCAUACGAAACG
GAGAAGCACAACGUGUGGGCGACCCAUGCCUGUGUCCCUACAGAC
CCUAACCCUCAGGAAAUUCAUCUUGAAAAUGUCACAGAAGAGUUU
AACAUGUGGAAAAACAACAUGGUGGAACAGAUGCACGAGGAUAUC
AUUUCCCUGUGGGACCAGAGUCUGAAACCAUGUGUCAAACUUACU
CCUCUGUGCGUGACUCUCCAGUGUACAAACUACGCACCCAACCUU
UUGAGUAAUAUGCGGGGCGAGCUCAAGCAGUGCAGUUUCAAUAUG
ACAACCGAAUUGAGAGACAAAAAACAGAAAGUAUACUCCCUCUUC
UACCGGCUGGACGUGGUGCAGAUCAAUGAGAACCAAGGAAAUAGA
AGCAACAACAGUAACAAGGAAUACCGGCUCAUAAAUUGCAAUACC
AGCGCUAUUACGCAGGCUUGCCCUAAGGUGAGCUUUGAGCCAAUC
CCGAUACAUUAUUGUGCCCCGGCAGGCUUCGCUAUACUGAAAUGC
AAGGAUAAGAAGUUUAAUGGGACAGGCCCUUGCCCUAGCGUUUCA
ACGGUCCAAUGUACCCACGGGAUCAAGCCCGUAGUGUCUACACAG
CUCCUGCUGAACGGCAGCCUGGCCGAAGAGGAGGUCAUAAUUAGG
AGCGAGAACAUAACUAACAACGCUAAAAACAUUCUCGUCCAGCUC
AAUACACCUGUGCAGAUCAACUGCACCCGGCCCAACAACAACACC
GUGAAGUCCAUUAGAAUUGGUCCGGGACAGGCAUUUUACUACUUC
GGAGAUAUAAUAGGCGAUAUCAGAAUGGCGCACUGUCAGGUGAGC
AAGGCCACCUGGAACGAGACCCUGGGCAAGGUGGUCAAACAGUUG
CGCAAGCACUUUGGGAACAACACCAUUAUUCGGUUUGCCCAGUCU
UCCGGCGGCGACCUUGAAGUGACCACUCAUAGCUUCAACUGUGGA
GGGGAGUUUUUCUAUUGCAAUACAUCAGGCCUGUUCAACUCUACA
UGGAUCUCAAAUACCAGUGUCCAGGGGUCAAAUUCCACCGGUAGC
AACGACAGCAUCGUCUUGCCUUGUCGAAUCAAGCAGAUCAUUAAU
AUGUGGCAGAGGAUUGGUCAGGCCAUGUACGCACCUCCAAUACAG
GGAGUCAUUCGGUGCGUCAGCAAUAUUACUGGAUUGAUCCUCACC
AGAGAUGGCGGGAGUACCAAUAGCACUACCGAAACUUUCCGCCCA
GGAGGAGGCGACAUGCGGGAUAAUUGGAGAUCAGAGCUGUAUAAG
UAUAAGGUGGUGAAAAUUGAACCCCUGGGAGUGGCGCCAACUAGA
UGUAAACGGCGAGUGGUUGGCCGGAGACGGCGGCGGAGAGCAGUG
GGGAUUGGCGCUGUCUCACUCGGUUUCCUGGGUGCUGCCGGCAGU
ACAAUGGGCGCCGCCAGCAUGACGCUCACAGUGCAGGCCCGGAAU
CUUCUUAGCGGAAUUGUGCAACAACAAAGCAAUCUGUUGAGAGCC
CCGGAACCGCAGCAACAUCUGUUGAAGGACACACAUUGGGGCAUC
AAGCAGCUGCAAGCUCGGGUUCUGGCUGUUGAGCAUUACCUGAGA
GACCAACAGCUGCUGGGCAUAUGGGGAUGCUCAGGAAAACUGAUC
UGCUGCACCAAUGUCCCAUGGAACAGCUCAUGGUCAAACAGGAAC
CUGAGCGAGAUCUGGGAUAACAUGACCUGGUUGCAGUGGGACAAA
GAAAUUAGCAAUUACACACAGAUCAUCUACGGCCUCCUGGAGGAA
AGCCAGAAUCAGCAGGAGAAAAAUGAGCAGGAUCUGCUUGCCCUU
GACUGA
SEQ ID NO: 6 (DNA sequence of WT_BG505)
ATGGACGCCATGAAGAGGGGACTTTGCTGTGTTCTTCTGCTGTGT
GGCGCCGTGTTTGTTAGCCCCGCTGGGGCCGGATCCAACCTGTGG
GTCACTGTGTATTATGGTGTGCCAGTGTGGAAGGATGCAGAGACA
ACACTCTTTTGCGCCTCCGACGCTAAAGCATACGAAACGGAGAAG
CACAACGTGTGGGCGACCCATGCCTGTGTCCCTACAGACCCTAAC
CCTCAGGAAATTCATCTTGAAAATGTCACAGAAGAGTTTAACATG
TGGAAAAACAACATGGTGGAACAGATGCACACCGATATCATTTCC
CTGTGGGACCAGAGTCTGAAACCATGTGTCAAACTTACTCCTCTG
TGCGTGACTCTCCAGTGTACAAACGTGACTAACAACATTACCGAC
GACATGCGGGGCGAGCTCAAGAATTGCAGTTTCAATATGACAACC
GAATTGAGAGACAAAAAACAGAAAGTATACTCCCTCTTCTACCGG
CTGGACGTGGTGCAGATCAATGAGAACCAAGGAAATAGAAGCAAC
AACAGTAACAAGGAATACCGGCTCATAAATTGCAATACCAGCGCT
ATTACGCAGGCTTGCCCTAAGGTGAGCTTTGAGCCAATCCCGATA
CATTATTGTGCCCCGGCAGGCTTCGCTATACTGAAATGCAAGGAT
AAGAAGTTTAATGGGACAGGCCCTTGCCCTAGCGTTTCAACGGTC
CAATGTACCCACGGGATCAAGCCCGTAGTGTCTACACAGCTCCTG
CTGAACGGCAGCCTGGCCGAAGAGGAGGTCATGATTAGGAGCGAG
AACATAACTAACAACGCTAAAAACATTCTCGTCCAGTTTAATACA
CCTGTGCAGATCAACTGCACCCGGCCCAACAACAACACCCGCAAG
TCCATTAGAATTGGTCCGGGACAGGCATTTTACGCTACAGGAGAT
ATAATAGGCGATATCAGACAGGCGCACTGTAACGTGAGCAAGGCC
ACCTGGAACGAGACCCTGGGCAAGGTGGTCAAACAGTTGCGCAAG
CACTTTGGGAACAACACCATTATTCGGTTTGCCAACTCTTCCGGC
GGCGACCTTGAAGTGACCACTCATAGCTTCAACTGTGGAGGGGAG
TTTTTCTATTGCAATACATCAGGCCTGTTCAACTCTACATGGATC
TCAAATACCAGTGTCCAGGGGTCAAATTCCACCGGTAGCAACGAC
AGCATCACCTTGCCTTGTCGAATCAAGCAGATCATTAATATGTGG
CAGAGGATTGGTCAGGCCATGTACGCACCTCCAATACAGGGAGTC
ATTCGGTGCGTCAGCAATATTACTGGATTGATCCTCACCAGAGAT
GGCGGGAGTACCAATAGCACTACCGAAACTTTCCGCCCAGGAGGA
GGCGACATGCGGGATAATTGGAGATCAGAGCTGTATAAGTATAAG
GTGGTGAAAATTGAACCCCTGGGAGTGGCGCCAACTAGATGTAAA
CGGCGAGTGGTTGGCCGGAGACGGCGGCGGAGAGCAGTGGGGATT
GGCGCTGTCTTCCTCGGTTTCCTGGGTGCTGCCGGCAGTACAATG
GGCGCCGCCAGCATGACGCTCACAGTGCAGGCCCGGAATCTTCTT
AGCGGAATTGTGCAACAACAAAGCAATCTGTTGAGAGCCCCGGAA
GCTCAGCAACATCTGTTGAAGCTGACAGTGTGGGGCATCAAGCAG
CTGCAAGCTCGGGTTCTGGCTGTTGAGAGATACCTGAGAGACCAA
CAGCTGCTGGGCATATGGGGATGCTCAGGAAAACTGATCTGCTGC
ACCAATGTCCCATGGAACAGCTCATGGTCAAACAGGAACCTGAGC
GAGATCTGGGATAACATGACCTGGTTGCAGTGGGACAAAGAAATT
AGCAATTACACACAGATCATCTACGGCCTCCTGGAGGAAAGCCAG
AATCAGCAGGAGAAAAATGAGCAGGATCTGCTTGCCCTTGACTGA
SEQ ID NO: 7 (AA sequence of BG505)
MDAMKRGLCCVLLLCGAVFVSPAGAGSNLWVTVYYGVPVWKDAET
TLFCASDAKAYETEKHNVWATHACVPTDPNPQEIHLENVTEEFNM
WKNNMVEQMHTDIISLWDQSLKPCVKLTPLCVTLQCTNVTNNITD
DMRGELKNCSFNMTTELRDKKQKVYSLFYRLDVVQINENQGNRSN
NSNKEYRLINCNTSAITQACPKVSFEPIPIHYCAPAGFAILKCKD
KKFNGTGPCPSVSTVQCTHGIKPVVSTQLLLNGSLAEEEVMIRSE
NITNNAKNILVQFNTPVQINCTRPNNNTRKSIRIGPGQAFYATGD
IIGDIRQAHCNVSKATWNETLGKVVKQLRKHFGNNTIIRFANSSG
GDLEVTTHSFNCGGEFFYCNTSGLFNSTWISNTSVQGSNSTGSND
SITLPCRIKQIINMWQRIGQAMYAPPIQGVIRCVSNITGLILTRD
GGSTNSTTETFRPGGGDMRDNWRSELYKYKVVKIEPLGVAPTRCK
RRVVGRRRRRRAVGIGAVFLGFLGAAGSTMGAASMTLTVQARNLL
SGIVQQQSNLLRAPEAQQHLLKLTVWGIKQLQARVLAVERYLRDQ
QLLGIWGCSGKLICCTNVPWNSSWSNRNLSEIWDNMTWLQWDKEI
SNYTQIIYGLLEESQNQQEKNEQDLLALD

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed:

1. A variant HIV Env polypeptide comprising a modification that disrupts at least one glycosylation site with respect to a native HIV Env polypeptide.

2. The polypeptide of claim 1, wherein said polypeptide comprises an amino acid sequence at least 97% identical to a sequence selected from the group consisting of SEQ ID NO: 2 and SEQ ID NO: 4.

3. The polypeptide of claim 1, wherein said polypeptide comprises a fragment of SEQ ID NO: 2 or SEQ ID NO: 4 comprising at least 75% of the full length of SEQ ID NO: 2 or SEQ ID NO: 4.

4. The polypeptide of claim 1, wherein the polypeptide elicits autologous neutralizing antibodies against HIV-1 in a mammal when administered to the mammal.

5. A nucleic acid molecule encoding the variant HIV Env polypeptide of any one of claims 1-4.

6. The nucleic acid molecule of claim 5, wherein the nucleic acid molecule comprises a sequence that is at least 98% identical to a sequence selected from the group consisting of SEQ ID NO: 1, SEQ ID NO: 3 and SEQ ID NO:5.

7. The nucleic acid molecule of claim 5, wherein the nucleic acid molecule comprises a fragment of SEQ ID NO: 1, SEQ ID NO: 3 or SEQ ID NO:5 comprising at least 75% of the full length of SEQ ID NO: 1, SEQ ID NO: 3 or SEQ ID NO:5.

8. The nucleic acid molecule of claim 5, wherein the nucleic acid molecule is selected from the group consisting of DNA and RNA.

9. A host cell comprising the nucleic acid of any one of claims 5-8.

10. A composition comprising the polypeptide of any one of claims 1-4.

11. The composition of claim 10, wherein the composition is a protein complex or a fusion protein.

12. A composition comprising the nucleic acid molecule of any one of claims 5-8.

13. The composition of claim 12, wherein the composition is selected from the group consisting of a virus-like particle, a nanoparticle, and a multimer.

14. The composition of claim 12, wherein the composition comprises a pharmaceutically acceptable carrier.

15. A method of stimulating an immune response in a subject in need thereof, comprising administrating to the subject an effective amount of a polypeptide of any one of claims 1-4 or a composition of any one of claims 10-11.

16. A method of stimulating an immune response in a subject in need thereof, comprising administrating to the subject an effective amount of a nucleic acid molecule of any one of claims 5-8, the host cell of claim 9 or a composition of any one of claims 12-14.

17. A method of eliciting autologous neutralizing antibodies against HIV-1 in a subject in need thereof, comprising administrating the subject an effective amount of a polypeptide of any one of claims 1-4 or a composition of any one of claims 10-11.

18. A method of eliciting autologous neutralizing antibodies against HIV-1 in a subject in need thereof, comprising administrating the subject an effective amount of a nucleic acid molecule of any one of claims 5-8, the host cell of claim 9 or a composition of any one of claims 12-14.

19. A method of treating or preventing HIV infection in a subject in need thereof, comprising administering to the subject a therapeutically effective amount of the polypeptide of any one of claims 1-4 or a composition of any one of claims 10-11.

20. A method of treating or preventing HIV infection in a subject in need thereof, comprising administering to the subject a therapeutically effective amount of the nucleic acid molecule of any one of claims 5-8, the host cell of claim 9 or a composition of any one of claims 12-14.
